(19) United States
(12) Patent Application Publication
(10) Pub. No.: US 2025/0262530 A1
(43) Pub. Date: Aug. 21, 2025

(54) APPARATUS, METHODS, AND SYSTEMS FOR CREATING A SENSORY IMMERSION THEATER

(71) Applicant: Jeffrey Mutschler TORTORICE, Coronado, CA (US)

(72) Inventor: Jeffrey Mutschler TORTORICE, Coronado, CA (US)

(21) Appl. No.: 19/057,889

(22) Filed: Feb. 19, 2025

Related U.S. Application Data
(60) Provisional application No. 63/555,542, filed on Feb. 20, 2024.

(51) Int. Cl. A63F 13/25; A61M 21/00; A61M 21/02

(52) U.S. Cl. CPC A63F 13/25; A61M 21/02; A61M 2021/0022; A61M 2021/0027; A61M 2021/005

(57) ABSTRACT

A sensory immersion theater (SIT) system is disclosed that includes a seating device having a left side and a right side, a display in front of the seating device; a projector or a light emitting diode for displaying content from a media device onto the display, and the media device sending stereo audio outputs and video outputs for visual stimuli (VS). The SIT system has a first transducer and a second transducer, the first transducer coupled to left side of the chair and the second transducer coupled to the right side of the chair; and an audio delivery device having a first audio output and a second audio output, wherein psycho-acoustic stimuli (PAS) is generated from the stereo audio outputs in the form of a PAS signal and vibrational acoustic stimuli (VAS) is generated from the stereo audio outputs in the form of a VAS signal, and a binaural output.

This application claims priority to Provisional Application No. 63/555,542, which is and its disclosure are incorporated herein in its entirety.

### BACKGROUND OF THE INVENTION

### 1. Field of the Invention

The present invention relates to apparatus, systems and methods for creating a sensory immersive experience for therapeutic, medical, gaming, and other applications.

### 2. Background

Sound may be heard through audio and playback through frequencies heard by the human ear. Sound may also be felt or observed visually through vibration and synchronized visual stimuli. Video is typically seen using the human eye.

Systems are well known that use audio, video and vibration to recreate or simulate real world environments. For example, virtual reality (VR) and associated technology are prevalent in the gaming, computing, and movie industries. In VR, simulations of real-world experiences may be created including those in nature, as between digital components, and the like.

Regardless of the technology, each uses speakers to recreate audio sounds and typically displays to provide video output to recreate the real world through a computer simulation. Such systems may also use acoustic and spatial technologies to make sounds applicable to a particular environment.

The displays and speakers in these computer systems may also employ sensors to enhance the audio and video experiences. In other known systems, video content may be displayed three dimensionally and the audio may be delivered over multiple channels.

Other systems also include mixed reality and virtual reality simulators used for training, entertainment, or educational reasons. The simulators are intended to simulate a three-dimensional world made up of digital audio sounds and computer created graphics.

As the development of these type systems continues, their applications can also be found in entertainment wellness. Therapy, and medicine. For example, systems are known to include vibrating chairs that use various vibrating frequencies and speaker systems, such as massage chairs. Typically, the vibration is provided by a combination of electrical rotating gears and wheels. Other induction devices may also be used. Other chairs or seats for gaming or in movie theaters also may use these vibrating techniques.

Other chairs that may be used in wellness and therapy use sound from transducers to create vibration The sound in these transducers may emit frequency vibrations that impact through sound waves. Yet, no known system uses a combination of audio, video and transducer technology that creates a sensory immersion experience utilizing a curved display screen or geodesic dome environment with various specific frequencies separated to each destination, such as a set of transducers or a set of headphones at the same time or simultaneously.

### SUMMARY

A sensory immersion theater (SIT) system is disclosed that includes a seating device having a left side and a right side; a display in front of the seating device; a projector or a light emitting diode for displaying content from a media device onto the display, the media device sending stereo audio outputs and video outputs for visual stimuli (VS); and a first transducer and a second transducer, the first transducer coupled to left side of the chair and the second transducer coupled to the right side of the chair. The system also includes an audio delivery device having a first audio output and a second audio output, wherein psycho-acoustic stimuli (PAS) is generated from the stereo audio outputs in the form of a PAS signal and vibrational acoustic stimuli (VAS) is generated from the stereo audio outputs in the form of a VAS signal, and a binaural output. The binaural output being wherein the PAS signal is separated into a first PAS input and a second PAS input that are each delivered to the first and second audio inputs, respectively and wherein the VAS signal is separated into a first VAS input and a second VAS input that are each delivered to the first and second transducers, respectively. The system may also include the seating device having a gap separating the left side from the right side. The display may be modular or concave.

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 shows an environment for use with a sensory immersion theater (SIT) system in accordance with an embodiment.

FIG. 2 shows a sensory immersion theater (SIT) in an embodiment.

FIG. 3 shows a timeline of various applications and treatments of a sensory immersion theater (SIT) in an embodiment.

FIG. 4 shows a process flow of a method using the SIT in an embodiment.

FIG. 5 shows a seating device to be used with the SIT systems in an embodiment.

FIG. 6 shows the SIT system in accordance with an embodiment.

### DETAILED DESCRIPTION

Each of the additional features and teachings disclosed below can be utilized separately or in conjunction with other features and teachings to provide a device, system, and/or method for content delivery and control. Representative examples of the present invention, which utilize many of these additional features and teachings both separately and in combination, will now be described in further detail with reference to the attached drawings. This detailed description is merely intended to teach a person of skill in the art further details for practicing preferred aspects of the present teachings and is not intended to limit the scope of the invention. Therefore, combinations of features and steps disclosed in the following detail description may not be necessary to practice the invention in the broadest sense and are instead taught merely to particularly describe representative examples of the present teachings.

Moreover, the various features of the representative examples and the dependent claims may be combined in ways that are not specifically and explicitly enumerated to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for original disclosure, as well as for the purpose of restricting the claimed subject matter.

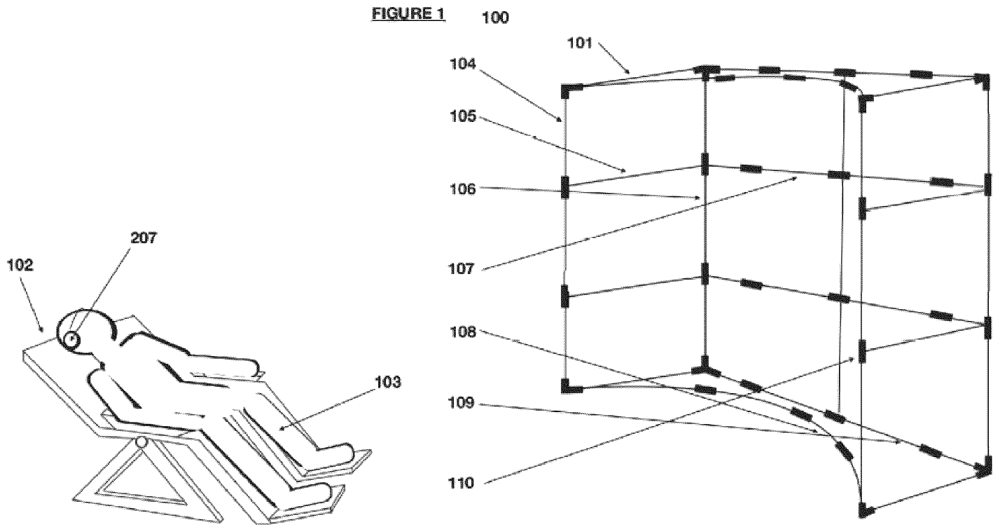

Devices, methods, and systems are described for a sensory immersion theater (SIT) that may be used for medical, gaming, or other applications, which may include a seating device, display, transducers, a projector, a video/audio media device, and an audio delivery apparatus. In some embodiments, the seating device may be a zero-gravity seating device or chair that may recline. In other embodiments, the seating device may be as shown in FIG. 1. The display may be substantially two-dimensional or substantially three dimensional. In some embodiments, the display may be a projector screen or a curved (concave) LED display. The audio delivery device may be a corded or cordless headphones or earbuds or wireless or wired headphones. The projector may be a light emitting diode or a stroboscopic headset in some embodiments.

In one embodiment, the SIT may use various stimuli in combination. It should be noted that the various stimuli may be changed in any combination. In one embodiment, the stimuli may include a psycho-acoustic stimuli (PAS) through transducers electronically coupled to the seating device, vibrational acoustic stimuli (VAS) through the audio delivery device, and visual stimuli (VS) on the display in one or more combinations. In one embodiment, the stimuli may be VAS+PAS+VS with a concave screen that is at least ten feet wide including the separated quadrants of acoustic delivery/vibro-acoustic delivery.

In one embodiment, a minimal distance ratio between viewers head and the screen width may be 2:3. In other embodiments, a distance ratio between viewers head and the screen width may be 1:2.

In one embodiment, the PAS may be audio files. In some embodiments, the VAS may be sound waves from, for example, a transducer. The VAS, PAS, and/or VS may be used together to bring a person's central nervous system into a state of alignment. In one embodiment, a state of alignment may be a Mind Body Coherence via brainwave entrainment.

In one embodiment, the stereo audio outputs from the media server may be separated between VAS and PAS, which may further be separated into four separate inputs. The PAS may include an input to the audio delivery device (headphones), which is divided between the right side and the left side of the audio delivery device and a second input to the VAS delivery devices, such as to transducer coupled to the right side and left side of a seating device.

The SIT includes separation of each input to the two seating device transducers (Left and Right) and the headphone stereo field (Left and Right) with corresponding synchronized visuals that are audio reactive. This method and system may create a binaural experience for the central nervous system that induces an entrained brainwave state that may be therapeutic in a variety of ways, including relaxation, deep Delta State rest, parasympathetic state, reduce anxiety, bilateral engagement for EMDR therapy, psychedelic assisted therapy, exposure therapy, brain spotting therapy, and general mind body wellness and coherence with corresponding synchronized audio reactive visual effects that create a real-time visual representation of the audio frequencies (e.g., cymatics visualizer effect).

In one embodiment, a zero gravity reclining seating device may have two transducers that have dedicated frequencies for each side. In one embodiment, the seating device may be split so that the right side of the body rests on one side of the seating device and the left side of the body rests on the other side of the seating device. In one example, the transducer attached to the left side of the seating device may have a frequency of 396 Hz. The other transducer attached to the right side of the seating device may have a frequency of 405 Hz. In this example, the headphones attached to the seating device may have dedicated frequencies for each ear in a stereo field. The left side of the headphones may have a frequency of 123 Hz. The right side of the headphones will have a frequency of 132 Hz. The format for the frequencies will remain relative, a 9 Hz differential with these specific frequencies, but the relative differential frequency can change.

In an embodiment, a format example may be as follows. Theta Entrainment (hour long program) will introduce Alpha frequencies with a variance of 9 Hz for a duration of 15 minutes then slowly transition down to Theta frequencies with a variance of 6 Hz for a duration of 30 minutes then slowly transition back up to Alpha frequencies with a variance of 9 Hz for a duration of 5 minutes then slowly transition up to Beta frequencies with a variance of 23 Hz or a duration of 5 minutes then slowly transition up to Gamma frequencies with a variance of 40 Hz or a duration of 5 minutes.

In one embodiment, the VAS may include a first input to a first transducer coupled to the seating device and a second input to a second transducer coupled to the seating device. In one embodiment, the first and second transducers are located underneath the seating device on opposing sides In one embodiment, the first and second input VAS inputs may have the same frequency. In other embodiments, the first and second input VAS inputs may have different frequencies.

In one embodiment, the frequency difference between the PAS inputs may be the same as the frequency difference between the VAS inputs. In other embodiments, the frequency difference between the PAS inputs may be different than the frequency difference between the VAS inputs. It should be noted that the frequency of the VAS and PAS inputs may be reversed over a period of time during an application. For example, the frequencies may be reversed every 30 seconds.

The visual frame rate or strobe rate of the VS may be the same as or different from frequency differential between the PAS and/or VAS inputs in an embodiment.

In some embodiments, the VS may include any images, video, or other data viewed using the eyes. In one embodiment, a combination of the VS, VAS, and PAS may bring a viewer into a state of relaxation or reflection or processing trauma, thereby providing treatment. In some embodiments, medications, herbs, or the like may be used in combination with the SIT. These may include psylocibin, ketamine, MDMA (3,4-Methyl enedioxy methamphetamine), 5MEO DMT (5-methoxy-N, N-Dimethyltriptamine), LSD, Ibogain, and/or other psychedelics or mind altering substances. The SIT may be used in the treatment of PTSD, depression, anxiety, pain, stress, memory loss, Alzheimer's, dementia, Traumatic Brain Injury, Suicidal Ideation, autism, social anxiety (agoraphobia), and/or cellular regeneration.

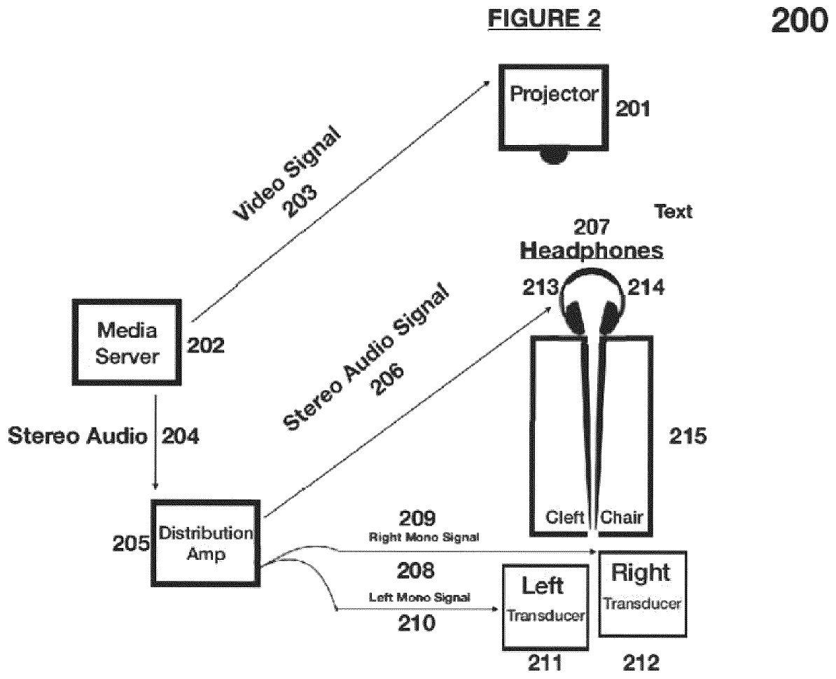

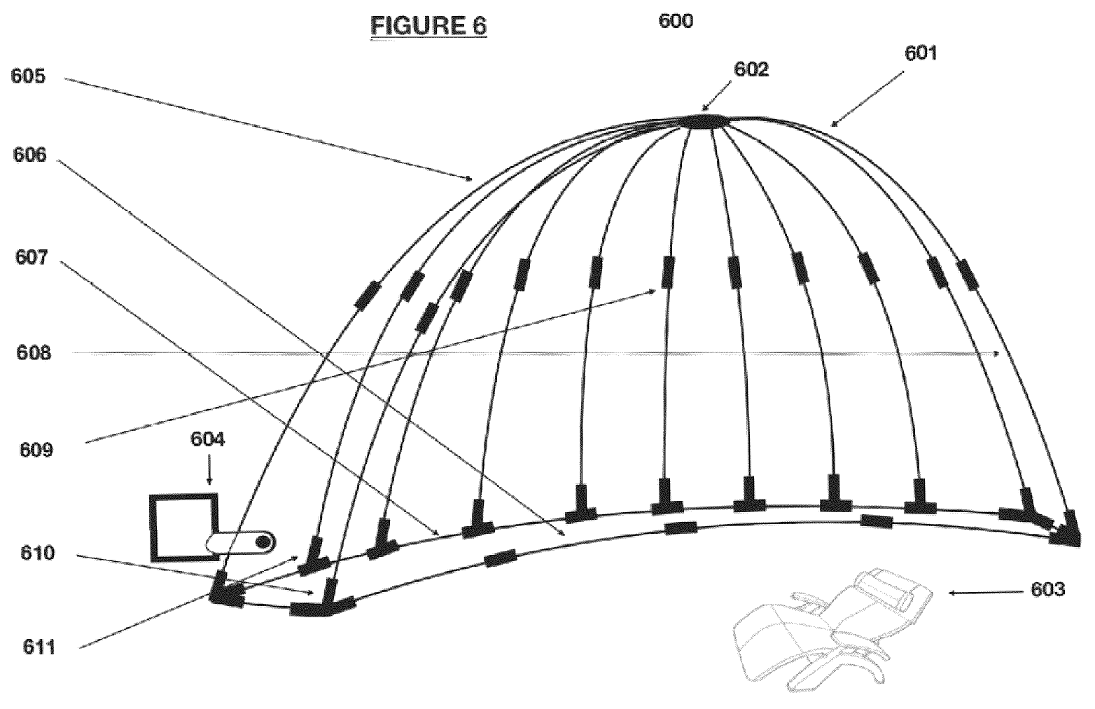

FIG. 1 shows an environment 100 for and FIG. 2 shows a sensory immersion theater (SIT) system in accordance with an embodiment. The environment 100 may include a display 101 and a seating device 102, which may be substantially the same as seating device 501. The seating device 102 may be used by a human 103 wearing an audio delivery device 207. The display 101 may be a screen projector in an embodiment. The display 101 may be concave projection screen in an embodiment. As shown in FIG. 6, the display may also be a shell or dome 601. In other embodiments, an LED display may be used. The seating device 102 may be a zero-gravity seating device in an embodiment. The size of the display 101 may vary. In one embodiment, the display 101 may have a width of approximately 180 inches and a height of approximately 101 inches. In other embodiments, the display may have a width of approximately 140 inches and a height of approximately 120 inches. The display 101 may include stretchable fabric screen that bends around the viewers peripheral vision to encompass their field of vision. A distance between the device 102 and the display 101 may be 8-10 feet in front of the seating device 102.

In one embodiment, the display 101 may be a stretchable screen canvas. The display 101 may include vertical rods 104, horizontal side rods 105, vertical back rods 106, horizontal back rods 107, curved front rods 108, three way corner couplers 109, and rod couplers 110. In one embodiment, the length and diameter of each of the rods may vary by the size of the display 101.

In one embodiment, the display 101, as shown in FIG. 1, may be a modular system that may include a frame with a weighted base (e.g., 50 lbs. on each side) to ensure stability. The frame may include: 8 flexible connecting tubes (for top and bottom of concave portion of the screen frame); 8 Vertical connecting tubes (the upright side supports—4 each side); 4 Z axis braces; 8 Rear cross braces; 8 corner couplers; and 16 straight couplers.

As shown in FIG. 2, the SIT system 100 may include a display device 201, such as a projector, coupled to a computing device 202 via a network connection 203. In some embodiments, the computing device 202 may be a media server. The network connection 203 may be wired or wireless. The computing device 202 may be configured to deliver an audio and/or video signal. In one embodiment, the computing device 202 delivers a video signal via connection 203 to the display device 201. The display device 201 may then display the video signal at a determined pixel density, resolution, and frame rate on display 101.

In one embodiment, the computing device 202 may deliver an audio signal 204 over a wired or wireless network connection to an audio amplifier 205. The output of the amplifier 205 may be separated into at least two outputs 206 or 208. In some embodiments, the computing device 202 may be a media player or media server.

In one embodiment, output 206 is further separated into PAS inputs 213, 214 of audio delivery device 207. In one embodiment, output 208 is further separated into VAS inputs 209 and 210 of transducers 212 and 211, respectively. Transducers 211 and 212 may also be configured on opposing sides of the seating device 215. Seating device 215 may be substantially the same as seating device 501

In one embodiment, transducers 2118 and 212 may be approximately 90-Watt transducers. In one embodiment, the transducers 211 and 212 may vibrate the seating device 215 in a haptic stereo field.

In one embodiment, the display device 201 may have a short throw capability with 1920×1080 resolution and 16:9 Aspect Ratio. The distance between the display device 201 and the display 101 may be approximately 10 feet or more in some embodiments. In other embodiments, 4k resolution may be used. In other embodiments, a curved LED screen may be used.

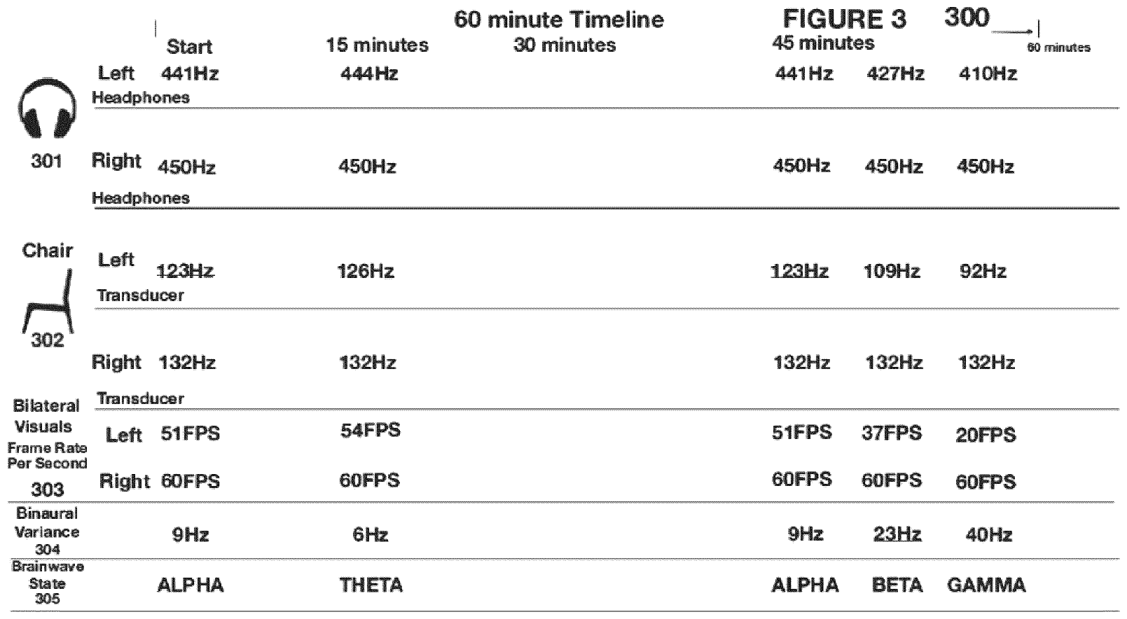

FIG. 3 shows a timeline 300 of various applications and treatments of a sensory immersion theater (SIT) in an embodiment. The timeline 300 shows changes in the PAS inputs 301, VAS inputs 302, and VS 303 for an application lasting about 60 minutes. A brainwave state 305 may be measured in an embodiment. The brainwave states may be as follows.
Gamma—30 Hz and above
Beta—13 Hz-30 Hz
Alpha—8 Hz-12 Hz
Theta—4 Hz-7 Hz
Delta—1 Hz-3 Hz

FIG. 3 also shows the Binaural Variance, which is the difference between the frequency in the left headphone and the right headphone of the audio delivery device 207.

It should be noted that the frequency changes between 45 minutes and 60 minutes in FIG. 3 change approximately every 5 minutes. FIG. 3 also shows the Bilateral Visuals, which are generally separation of the left and right sides of the screen to be synchronized with the bilateral variance in Frame Rate Per Second according to the psycho acoustic and vibro-acoustic input frequencies.

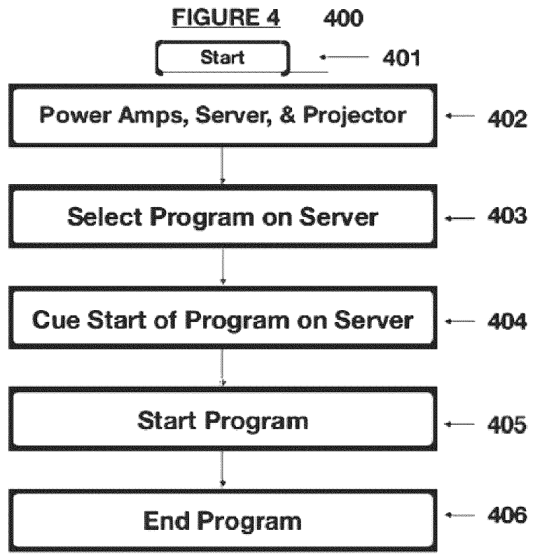

FIG. 4 shows a process flow 400 of a method using the SIT in an embodiment. At step 401 the process starts. At step 402, the power to the transducer amplifiers, media servers and display device is initiated. At step 403, the desired program is selected on the server. At step 404, the program is started on the server, Exemplary programs are shown in FIG. 3. At step 405, the program is started and at step 406 the program ends.

Below are additional examples of using the SIT.
30 min Gamma Session @ 40 Hz

### Program:

Beta starts at 23 Hz and sustains for 5 minutes and ascends to 40 Hz over the last 2 minutes

### Transducer Inputs

Left=220 Hz
Right=260 Hz

### Headphone Inputs

Left=110 Hz
Right=150 Hz

### Bilateral Visuals

Left=20 FPS
Right=60 FPS
30 min Schumann Session @ 7.83 Hz+30 min Gamma Session@ 40 Hz

### Program:

Beta starts at 7.83 Hz and sustains for 30 minutes and ascends to 40 Hz over the last 5 minutes

### Schumann Frequency (THETA)

### Transducer Inputs

Left=102.17 Hz
Right=110 Hz

### Headphone Inputs

Left=55 Hz
Right=62.83 Hz

### Bilateral Visuals

Left=52.17 FPS
Right=60 FPS

### Gamma

### Transducer Inputs

Left=220 Hz
Right=260 Hz

### Headphone Inputs

Left=110 Hz
Right=150 Hz

### Bilateral Visuals

Left=20 FPS
Right=60 FPS
60 min Alpha Session @ 9 Hz+30 min Theta Session@ 6 Hz

### Program:

Alpha starts at 9 Hz and sustains for 10 minutes then descends to 6 Hz over the last 5 minutes then sustains for 20 min then ascends to 9 Hz over the last 5 minutes and sustains for 10 minutes then ascends to 40 Hz for last 20 minutes, which equals a total of one hour.

### Alpha

### Transducer Inputs

Left=396 Hz
Right=405 Hz

### Headphone Inputs

Left=123 Hz
Right=132 Hz

### Bilateral Visuals

Left=423 FPS
Right=432 FPS

### Theta

### Transducer Inputs

Left=126 Hz
Right=132 Hz

### Headphone Inputs

Left=110 Hz
Right=116 Hz

### Bilateral Visuals

Left=54 FPS
Right=60 FPS

### Gamma

### Transducer Inputs

Left=220 Hz
Right=260 Hz

### Headphone Inputs

Left=110 Hz
Right=150 Hz

### Bilateral Visuals

Left=20 FPS
Right=60 FPS

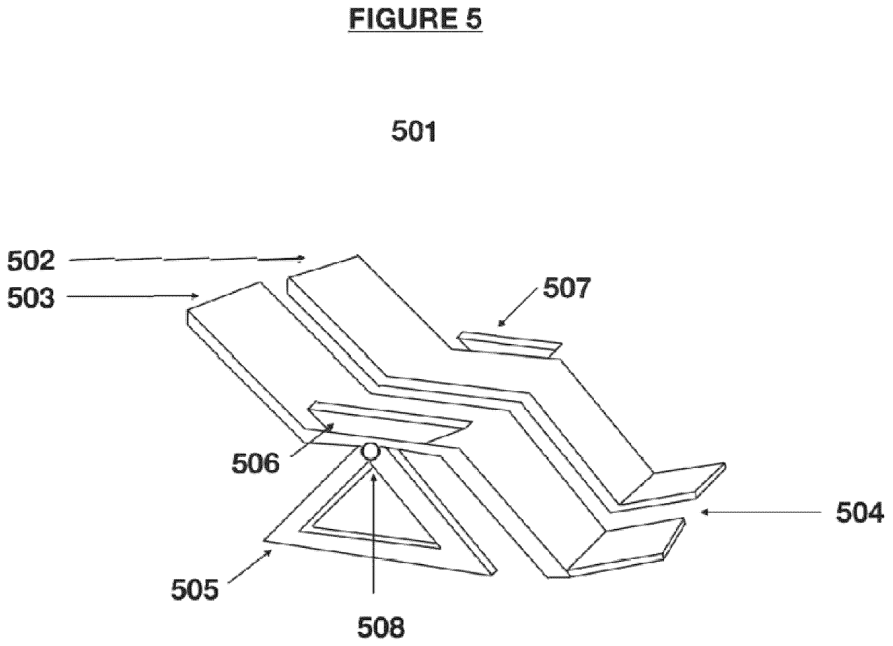

FIG. 5 is a seating device 501 in an embodiment. The seating device 501 may include a left side 502 and a right side 503. In some embodiments, the left side 502 may be occupied substantially by the left side of a human body. The right side 503 may be occupied substantially by the right side of a human body. The seating device 501 may include a gap 504. The gap 504 makes a clear separation between the different sides of the body such no interference or vibratory cross contamination between the two sides from any of the audio, video, or vibrational signals. In an embodiment, the gap 504 creates a complete separation between sides 502 and 503. Each of the sides 502 and 503 may rest individually on a stand 505. Each stand may have a swivel adjustments device 508 that enables each side of the chair 506, 507 to rotate between various positions. The seating device 501 may also include arm rests 505 and 506.

FIG. 6 is the SIT system 600 in an embodiment. The system 600 may include a display screen 601 and a seating device 603 The seating device 603 may be substantially the same as seating device 501.

In one embodiment, the display screen 601 may be coupled to a vacuum seal rear cover and reverse fan 604 to keep the screen pulled in and taught. The display screen 601 may be a stretchable screen canvas. The display screen 601 may include curved vertical rods 605 that are each coupled to a crown coupler 602. The screen 601 may also include curved rod front base rods 606, curved back base rods 607, side base rods 608, rod couplers 609, three-way rod couplers 610 and two-way rod couplers 611.

The foregoing description of the preferred embodiments of the present invention has been presented for purposes of illustration and description. It is not intended to be exhaustive or to limit the invention to the precise form or to exemplary embodiments disclosed. Obviously, many modifications and variations will be apparent to practitioners skilled in this art. Similarly, any process described step might be interchangeable with other steps in order to achieve the same result. The embodiment was chosen and described in order to best explain the principles of the invention and its best mode practical application, thereby enabling others skilled in the art to understand the invention for various embodiments and with various modifications as are suited to the particular use or implementation contemplated. It is intended that the scope of the invention be defined by the claims appended hereto and their equivalents. Reference to an element in the singular is not intended to mean “one and only one” unless explicitly so stated but rather means “one or more.” Moreover, no element, component, nor method step in the present disclosure is intended to be dedicated to the public regardless of whether the element, component, or method step is explicitly recited in the following claims. No claim element herein is to be construed under the provisions of 35 U.S.C. Sec. 112, sixth paragraph, unless the element is expressly recited using the phrase “means for . . . .”

It should be understood that the various techniques described herein may be implemented in connection with hardware or software or, where appropriate, with a combination of both. Thus, the methods and apparatus of the presently disclosed subject matter, or certain aspects or portions thereof, may take the form of program code (i.e., instructions) embodied in tangible media, such as hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be affected across a plurality of devices. Such devices might include personal computers, network servers, and handheld devices, for example.

Furthermore, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present invention in any way. It is also to be understood that the steps and processes recited in the claims need not be performed in the order presented.

1. A sensory immersion theater (SIT) system, comprising:
a seating device having a left side and a right side;
a display in front of the seating device;
a projector or a light emitting diode for displaying content from a media device onto the display, the media device sending stereo audio outputs and video outputs for visual stimuli (VS);
a first transducer and a second transducer, the first transducer coupled to left side of the chair and the second transducer coupled to the right side of the chair; and
an audio delivery device having a first audio output and a second audio output, wherein psycho-acoustic stimuli (PAS) is generated from the stereo audio outputs in the form of a PAS signal and vibrational acoustic stimuli (VAS) is generated from the stereo audio outputs in the form of a VAS signal, and a binaural output, the binaural output being wherein the PAS signal is separated into a first PAS input and a second PAS input that are each delivered to the first and second audio inputs, respectively and wherein the VAS signal is separated into a first VAS input and a second VAS input that are each delivered to the first and second transducers, respectively

2. The system of claim 1, wherein the seating device has a gap separating the left side from the right side.

3. The system of claim 1, wherein the display is concave.

4. The system of claim 1, wherein the display is modular.

5. The system of claim 1, wherein the binaural output has a differential of about 9 Hz.

6. The system of claim 1, wherein the seating device is a zero gravity chair.

7. The system of claim 1, wherein the display is a partial shell.

8. The system of claim 1 further comprising an amplifier coupled to the first VAS input and the second VAS input.

9. The system of claim 1, further comprising one or more brain states.

10. The system of claim 1, wherein a minimal distance ratio between a viewer's head and the display may be 2:3.

11. A sensory immersion theater (SIT) system, comprising:
a seating device having a left side and a right side;
a media device sending stereo audio outputs and video outputs for visual stimuli (VS);
a first transducer and a second transducer, the first transducer coupled to left side of the chair and the second transducer coupled to the right side of the chair; and
an audio delivery device having a first audio output and a second audio output, wherein psycho-acoustic stimuli (PAS) is generated from the stereo audio outputs in the form of a PAS signal and vibrational acoustic stimuli (VAS) is generated from the stereo audio outputs in the form of a VAS signal, and a binaural output, the binaural output being wherein the PAS signal is separated into a first PAS input and a second PAS input that are each delivered to the first and second audio inputs, respectively and wherein the VAS signal is separated into a first VAS input and a second VAS input that are each delivered to the first and second transducers, respectively.

12. The system of claim 11, wherein the seating device has a gap separating the left side from the right side.

13. The system of claim 11, including a display wherein the display is concave.

14. The system of claim 11, including a display wherein the display is modular.

15. The system of claim 11, wherein the binaural output has a differential of about 9 Hz.

16. The system of claim 11, wherein the seating device is a zero gravity chair.

17. The system of claim 11, including a display wherein the display is a partial shell.

18. The system of claim 11 further comprises an amplifier coupled to the first VAS input and the second VAS input.

19. The system of claim 11, further comprising one or more brain states.

20. The system of claim 11, including a display and wherein a minimal distance ratio between a viewer's head and the display may be 2:3.
